(19) United States
(12) Patent Application Publication
(10) Pub. No.: US 2024/0312621 A1
(43) Pub. Date: Sep. 19, 2024

(54) SYSTEM AND A METHOD FOR MONITORING ACTIVITIES OF AN OBJECT

(71) Applicant: Logistics and Supply Chain MultiTech R&D Centre Limited, HK (HK)

(72) Inventors: Kin Keung LEE, HK (HK); Ka Yau Lau, HK (HK); Chun Fai CHEUNG, HK (HK); To Bun NG, HK (HK); Ka Lun FAN, HK (HK); Chun Wai LEUNG, HK (HK); Chun On WONG, HK (HK)

(21) Appl. No.: 18/184,890

(22) Filed: Mar. 16, 2023

(51) Int. Cl. G16H 40/67; G06T 7/73; G06V 20/52

(52) U.S. Cl. CPC G16H 40/67; G06T 7/73; G06V 20/52; G06T 2200/04; G06T 2207/10012; G06T 2207/10028; G06T 2207/20092; G06T 2207/30004; G06T 2207/30204; G06V 2201/03

(57) ABSTRACT

A system and a method for monitoring activities of an object. The method comprises the steps of: providing a depth image capturing at least a part of the object and an item of supporting furniture, wherein the item of supporting furniture is provided with a support surface arranged to physically support the object to be disposed thereon, and the object is movable relative to the item of supporting furniture; processing the depth image to determine an activity of the object and the item of supporting furniture including the support surface being captured in the depth image; analyse the objects posture and location using AI models that are trained using depth images and/or using the skeleton of the object; analyses the intention of the object's movement; and generating an alert upon a determination of the activity of the object being identified as a risky activity.

### TECHNICAL FIELD

The invention relates to a system and a method for monitoring activities of an object, and particularly, although not exclusively, to a system for monitoring activities of a patient or an object requiring caregivers' attentions based on computer vision.

### BACKGROUND

In a hospital or an elderly home, it is common to find a large number of patients undergoing various forms of medical care and treatment of varying durations. Tagging labels may be used for identifying these patients as it is important to keep track of each patient to ensure correct medical care or security is administered by the hospital authority and medical staff. These labels may be provided with barcodes and text so that the label may be read or scanned by a barcode scanner. These labels may then be tided on the wrist of the patient, or simply attached to the patient with adhesives.

However, tagging devices worn by patients are only capable of tagging the patient and can only provide a location of the patient. To monitor activities of these patient, it can only be carried out in an in-person manner, or via surveillance cameras. Sometimes, patients may undertake risky activities and/or intention without actually moving to another position at the premises.

### SUMMARY OF THE INVENTION

In accordance with a first aspect of the present invention, there is provided a method for monitoring activities of an object, comprising the steps of: providing a depth image capturing at least a part of the object and an item of supporting furniture, wherein the item of supporting furniture is provided with a support surface arranged to physically support the object to be disposed thereon, and the object is movable relative to the item of supporting furniture; processing the depth image to determine an activity of the object and the item of supporting furniture including the support surface being captured in the depth image; and generating an alert upon a determination of the activity of the object being identified as a risky and/or intention.

In accordance with the first aspect, the 3D spatial sensor includes at least one of a 3D LiDAR module, a solid-state LiDAR module or an IR structure light sensor and stereo camera.

In accordance with the first aspect, the step of processing the depth image comprising the step of converting the depth image to point cloud data for further 3D analysis of the object and the item of supporting furniture so as to determine the activity of the object.

In accordance with the first aspect, the step of processing the depth image further comprising the step of identifying a location of the item of supporting furniture, including locating the support surface of the item of supporting furniture captured in the depth image.

In accordance with the first aspect, the step of identifying the location of the item of supporting furniture includes at least one of: identifying one or more machine-detectable markers each indicating a predetermined position of a feature of the item of supporting furniture; annotating the location of the item of supporting furniture by an operator; or determining the location of the item of supporting furniture using AI image recognition.

In accordance with the first aspect, the step of processing the depth image further comprising the step of identifying a status of the item of supporting furniture and/or other furniture detectable by one or more sensors and/or computer vision.

In accordance with the first aspect, the step of processing the depth image further comprising the step of identifying a position and/or a posture of the object based on machine learning and/or a skeleton of the object.

In accordance with the first aspect, the step of processing the depth image further comprising the step of predicting the risky activity performed by the object with reference to a tracked posture of the object captured in a single and/or a sequence of depth image(s) and the status of the furniture other than the supporting furniture.

In accordance with the first aspect, the step of processing the depth image further comprising the step of identifying a portion of the object being outside of the support surface to determine if the activity is risky based on a ratio of points in the point cloud representing the object staying on/above the support surface of the item of supporting furniture and outside of the support surface.

In accordance with the first aspect, the object is a patient or an object requiring caregivers' and/or other people's attentions.

In accordance with a second aspect of the present invention, there is provided a system of monitoring activities of an object, comprising: a 3D spatial sensor arranged to provide a depth image capturing at least a part of the object and an item of supporting furniture, wherein the item of supporting furniture is provided with a support surface arranged to physically support the object to be disposed thereon, and the object is movable relative to the item of supporting furniture; a processing module arranged to process the depth image to determine an activity of the object and the item of supporting furniture including the support surface being captured in the depth image; and a warning module arranged to generate an alert upon a determination of the activity of the object being identified as a risky activity.

In accordance with the second aspect, the depth image captured by a 3D spatial sensor includes stereo camera, 3D solid-state LiDAR, and light camera etc.

In accordance with the second aspect, the 3D spatial sensor includes at least one of a 3D LiDAR module, a solid-state LiDAR module, an IR structure light sensor and/or stereo camera.

In accordance with the second aspect, the depth image includes no RGB information.

In accordance with the second aspect, the processing module is arranged to convert the depth image to point cloud data for further analysis of the object and the item of supporting furniture so as to determine the activity of the object.

In accordance with the second aspect, the processing module includes an embedded computer or a cloud server in communication with the embedded computer.

In accordance with the second aspect, the processing module is arranged to identify a location of the item of supporting furniture, including to locate the support surface of the item of supporting furniture captured in the depth image.

In accordance with the second aspect, the processing module is arranged to identify a location of the item of supporting furniture by performing at least one of: identifying one or more machine-detectable markers each indicating a predetermined position of a feature of the item of supporting furniture; annotating the location of the item of supporting furniture by an operator; or determining the location of the item of supporting furniture using AI image recognition.

In accordance with the second aspect, the processing module is arranged to identify a status of the item of supporting furniture detectable by one or more sensor and/or computer vision.

In accordance with the second aspect, the one or more sensor includes a touch sensor, a motion sensor, an inertial measurement unit and/or a height sensor to measure the furniture's status and information.

In accordance with the second aspect, the processing module is arranged to identify a position and/or a posture of the object based on machine learning and/or a skeleton of the object.

In accordance with the second aspect, the processing module is further arranged to identify a posture and/or a position of the object based on a head and/or a shoulder of the object.

In accordance with the second aspect, the processing module is arranged to predict the risky activity performed by the object with reference to (i) a tracked posture of the object captured in a single and/or a sequence of depth image(s) and/or (ii.) the status of the furniture other than the supporting furniture.

In accordance with the second aspect, the processing module is further arranged to predict an intention or tendency of the object with reference of a level of activity performed by the object.

In accordance with the second aspect, the warning module is further arranged to generate the alert upon identifying that the object tends to fall from and/or to leave the support surface.

In accordance with the second aspect, the processing module is arranged to identify a portion of the object being outside of the support surface to determine if the activity is risky based on a ratio of points in the point cloud representing the object staying on/above the support surface of the item of supporting furniture and outside of the support surface.

In accordance with the second aspect, the activity is determined to be risky upon the ratio of points in the point cloud representing the object staying on/above the support surface of the item of supporting furniture and outside of the support surface exceeding a predetermined threshold.

In accordance with the second aspect, the processing module is further arranged to store a risk profile associated with a predetermined set of risky activity associated to postures and/or activities of the object.

In accordance with the second aspect, the object is a patient or an object requiring a caregiver's and/or other people's attentions.

In accordance with the second aspect, the warning module includes a client's device arranged to facilitate observation and monitoring of a status of object by a caregiver.

In accordance with the second aspect, the warning module is arranged to generate the alert upon detecting an activity performed by the object for a predetermined period of time.

In accordance with the second aspect, the support surface includes a bed surface or a chair surface.

### BRIEF DESCRIPTION OF THE DRAWINGS FOR THE INVENTION

Embodiments of the present invention will now be described, by way of example, with reference to the accompanying drawings in which:

FIG. 1 is a schematic diagram of a computer server which is arranged to be implemented as a system for monitoring activities of an object in accordance with an embodiment of the present invention.

FIG. 2 is a block diagram of a system for monitoring activities of an object in accordance with an embodiment of the present invention.

FIG. 3 is a flow diagram showing a method for monitoring activities of an object in accordance with an embodiment of the present invention.

FIG. 4A is an image of an example furniture item which may be recognized by the system of FIG. 2.

FIG. 4B is a depth image of the furniture item of FIG. 4A.

FIG. 5A is an image of a patient or target who performs a risky activity.

FIG. 5B is a depth image of the patient or target FIG. 5A.

FIG. 6 is an image of an example furniture item which is installed with additional sensors for event detection by the system of FIG. 2.

### DETAILED DESCRIPTION OF THE PREFERRED EMBODIMENT OF THE INVENTION

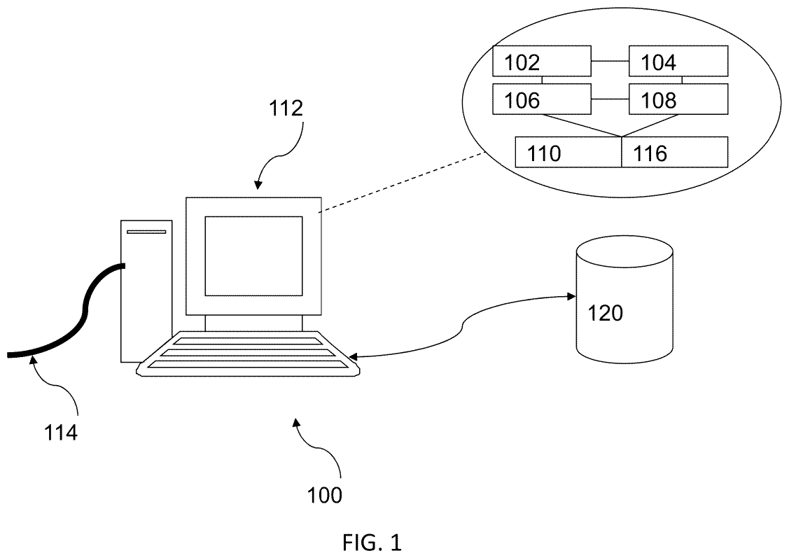

Referring to FIG. 1, an embodiment of the present invention is illustrated. This embodiment is arranged to provide a system of monitoring activities of an object, comprising: an 3D spatial sensor arranged to provide a depth image capturing at least a part of the object and an item of supporting furniture, wherein the item of supporting furniture is provided with a support surface arranged to physically support the object to be disposed thereon, and the object is movable relative to the item of supporting furniture; a processing module arranged to process the depth image to determine an activity of the object and the item of supporting furniture including the support surface being captured in the depth image; and a warning module arranged to generate an alert upon a determination of the activity of the object being identified as a risky activity.

In this example embodiment, the interface and processor are implemented by a computer having an appropriate user interface. The computer may be implemented by any computing architecture, including portable computers, tablet computers, stand-alone Personal Computers (PCs), smart devices, Internet of Things (IOT) devices, edge computing devices, patient/server architecture, “dumb” terminal/mainframe architecture, cloud-computing based architecture, or any other appropriate architecture. The computing device may be appropriately programmed to implement the invention.

The system may be used to help monitoring one or more moving targets, such as patients in a hospital and/or elderly home or objects requiring caregivers' attentions, and if necessary, warning or notifying the caregivers if the patients are in danger or difficult situations.

As shown in FIG. 1 there is a shown a schematic diagram of a computer system or computer server 100 which is arranged to be implemented as a system of monitoring activities of an object. In this embodiment the system comprises a server 100 which includes suitable components necessary to receive, store and execute appropriate computer instructions. The components may include a processing unit 102, including Central Processing Unit (CPUs), Math Co-Processing Unit (Math Processor), Graphic Processing Unit (GPUs) or Tensor processing unit (TPUs) for tensor or multi-dimensional array calculations or manipulation operations, read-only memory (ROM) 104, random access memory (RAM) 106, and input/output devices such as disk drives 108, input devices 110 such as an Ethernet port, a USB port, etc. Display 112 such as a liquid crystal display, a light emitting display or any other suitable display and communications links 114. The server 100 may include instructions that may be included in ROM 104, RAM 106 or disk drives 108 and may be executed by the processing unit 102. There may be provided a plurality of communication links 114 which may variously connect to one or more computing devices such as a server, personal computers, terminals, wireless or handheld computing devices, Internet of

Things (IOT) devices, smart devices, edge computing devices. At least one of a plurality of communications link may be connected to an external computing network through a telephone line or other type of communications link.

The server 100 may include storage devices such as a disk drive 108 which may encompass solid state drives, hard disk drives, optical drives, magnetic tape drives or remote or cloud-based storage devices. The server 100 may use a single disk drive or multiple disk drives, or a remote storage service 120. The server 100 may also have a suitable operating system 116 which resides on the disk drive or in the ROM of the server 100.

The computer or computing apparatus may also provide the necessary computational capabilities to operate or to interface with a machine learning network, such as neural networks, to provide various functions and outputs. The neural network may be implemented locally, or it may also be accessible or partially accessible via a server or cloud-based service. The machine learning network may also be untrained, partially trained or fully trained, and/or may also be retrained, adapted or updated over time.

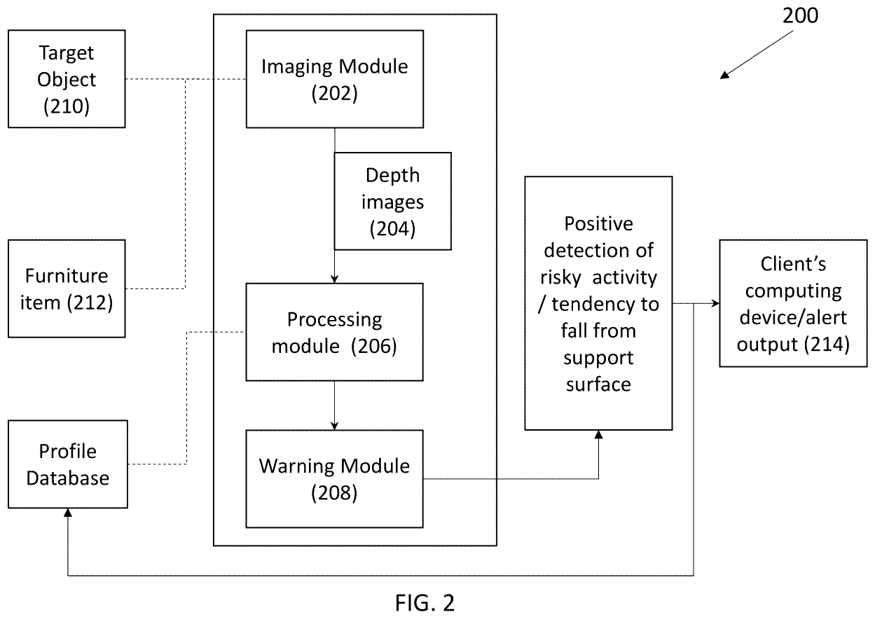

In accordance with a preferred embodiment of the present invention, with reference to FIG. 2, there is provided an embodiment of the system 200 monitoring activities of an object, such as a moving object. In this embodiment, the server 100 is used as part of a system arranged to receive images or spatial data captured by a 3D spatial sensor such as a depth camera, and to determine if the target object, such as a patient who should be staying on a bed surface, leaving the bed, falling or has a tendency to fall from the bed surface accidentally. In addition, the system may also provide a warning to a user of the system, such as a nurse or a caregiver, who may take appropriate action in response to the accident.

For example, the system may be used to monitor a group of patient in a hospital or elderly home, and 3D spatial sensors may be installed in different rooms for capturing the surface of one or more beds, each should be occupied by a patient or a tenant. By switching on the alert function, the system may help the caregiver to immediately response to any accident, or to take immediate administrative action if any patient leaves the bed for an unexpected period of time, upon receiving an alert provided by the system.

In this embodiment, the system 200 comprises a 3D spatial sensor, such as a depth camera and 3D LiDAR, for capturing depth images 204 including depth data or spatial information associated with a target object 210 captured by the 3D spatial sensor.

For example, the system is implemented as a 3D spatial sensor system for monitoring a predetermined area and for detecting activities of moving objects in that area. Preferably, the 3D spatial sensor system comprises a 3D spatial sensor, such as a 3D LiDAR, Solid-state LiDAR and/or Infrared (IR) structure light sensors and/or stereo camera etc. Advantageously, the monitoring/detection does not rely on RGB images and thereby privacy of the monitored object may be preserved.

Referring to FIG. 2, the system also comprises a processing module 206 for processing the depth images being acquired by the 3D spatial sensor/camera. Preferably, a computer system, such as embedded computer may be used to collect high resolution (for example VGA or even higher) depth data captured by the 3D spatial sensor, subsequently, the depth data may be converted to point cloud data for 3D analysis. The detection method may be implemented on an embedded computer locally, or remotely on a cloud server connected to the embedded computer, in which captured data may be transmitted to the cloud server for analysis.

In addition, a client computing device may be included for users such as nurses and caregivers can view and monitor the status of the bed, or other furniture item 212, and the patient 210. The client computing device may be provided as a separate computer system, such as a desktop computer, a laptop computer, a tablet computer or a smartphone, installed with appropriate patient software or application. Alternatively, the same client computing device may also be used as the processing module for processing the images/data captured by the 3D spatial sensor.

To further allow a caregiver to promptly provide support to the patient or to prevent accident such as the patient falling off from the bed or chair surface, a warning module 208 may be included in the system 200 to generate an alert 214 upon a determination of the activity of the object being identified as a risky activity, e.g. if it is expected that a patient may lose his balance from the bed surface if a large portion of his torso is no longer support by the bed surface. In this disclosure, the “level of activity” of the patient is analyzed so as to predict if the patient is prone to fall and/or tends to leave the bed surface accidentally/unexpectedly.

For example, the patient's software application may show a warning message 214 which may promptly alert the caregiver to pay attention to the risky activity performed by the patient, or may simply warn the patient to cease his activity to avoid an occurrence of accident, when the patient outreaches his arm and his upper torso from the bed surface and exceeds the safety threshold determined by the system.

Preferably, AI image/object recognition may be used to analysis the image/data captured by the 3D spatial sensor, such as but not limited to: furniture identifications, locating and status analysis; patient detection and locating; patient's posture, event and intent detection; data analysis for finding “level of activity” and predicting if the patient is prone to fall and/or tends to leave bed”, this can tailor a profile for the patient for future uses. In this example, the processing module may store a risk profile associated with a predetermined set of risky activity associated to postures and/or activities of the object.

In this example embodiment, the processing module is further arranged to predict an intention or tendency of the object with reference of a level of activity performed by the object, and the detection or successful detection may be stored in a profile database for profile optimization as shown in FIG. 2.

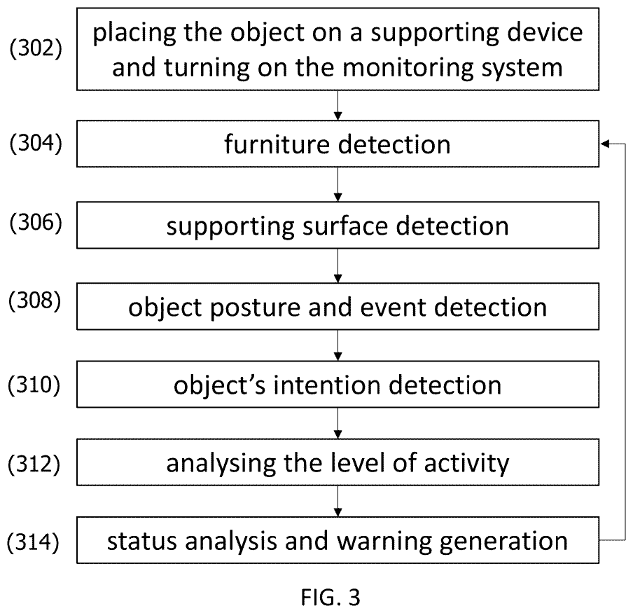

With reference to FIG. 3, there is shown an example operation flow of a method 300 for monitoring activities of an object, comprising the key steps of: providing a depth image capturing at least a part of the object and an item of supporting furniture, wherein the item of supporting furniture is provided with a support surface arranged to physically support the object to be disposed thereon, and the object is movable relative to the item of supporting furniture; processing the depth image to determine an activity of the object and the item of supporting furniture including the support surface being captured in the depth image; and generating an alert upon a determination of the activity of the object being identified as a risky activity.

At step 302, the user may place the object, such as a patient on a supporting device (chair and bed etc.) or surface of furniture and turn on the sensor and the monitoring system, at step 304, the system detects the furniture, such as bed or chair, or the surface of the furniture according to the “furniture detection” process, at step 306, the system further detects the supporting surface of the furniture according to the “supporting surface detection” process. At step 308, the system detects the patient's posture and event according to the “object posture and event detection” process. At step 310, the system further evaluates the patient's intention according to the “object's intention detection” process.

In addition, at step 312 the system may also record and analyse the “level of activity” and “if the patient is prone to fall and/or tends to leave bed”. Profile for individual object/patient can be built based on this, the system sensitivity can then be adjusted for the future use. Finally, the system analyses results of steps 304, 306, 308, 310 and 312, and sends out warning messages if necessary, at step 314, according to the “status analysis and warning generation” process. The system repeats steps 304 to 314 when the alert function is switched on by the caregiver.

The abovementioned processes, including the “furniture detection” process, at step 304, “supporting surface detection” process at step 306, “object posture and event detection” process at step 308 and “status analysis and warning generation” process at step 314 are further explain as follows.

In this example, by performing the “furniture detection”, the system recognises all the furniture and/or detecting their locations, which may be performed by one or more of the following methods, including: locations annotated in the system by the user or an operator of the system; AI training on the depth and/or point cloud image and machine learning to detect the furniture automatically; and/or landmarks being added to the furniture, they can be invisible to human eyes but detectable by the sensors. Since the location of landmark are known and can be found by the sensors and AI, the furniture location can be calculated using the point cloud data.

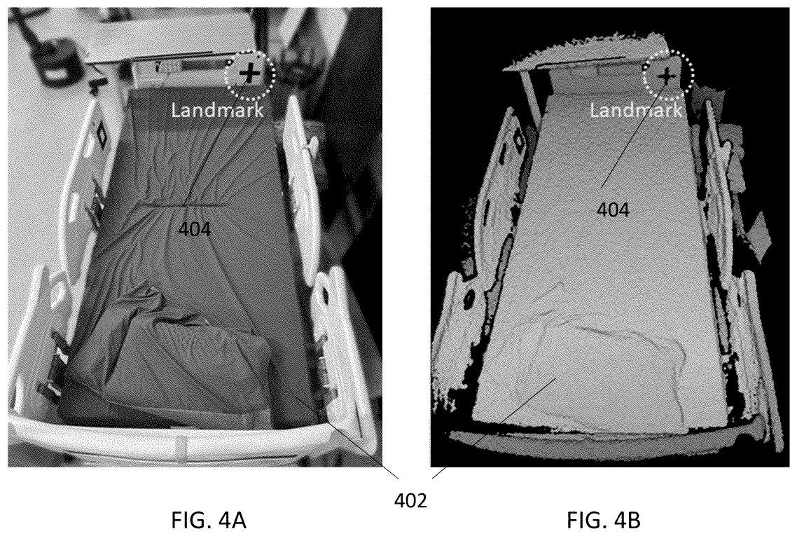

Referring to FIGS. 4A and 4B, the position of a bed 402 is determined by identifying a machine-detectable marker 404 which indicates a predetermined position of a feature of the item of the bed. In some alternative examples, multiple markers may be added to improve the accuracy of locating the furniture item, using computer vision.

Preferably, the processing module is arranged to identify a status of the item of supporting furniture detectable by one or more sensor and/or computer vision. For example, the status of the furniture can also be determined using AI or by adding sensors, such as door sensor or inertia/motion sensor to the furniture item. For example, when a door of a table/closet is touched/moved, it can be detected by using AI or adding sensors (for example touch sensor to detect if it is touched and inertial measurement unit (IMU) to detect if it is moved).

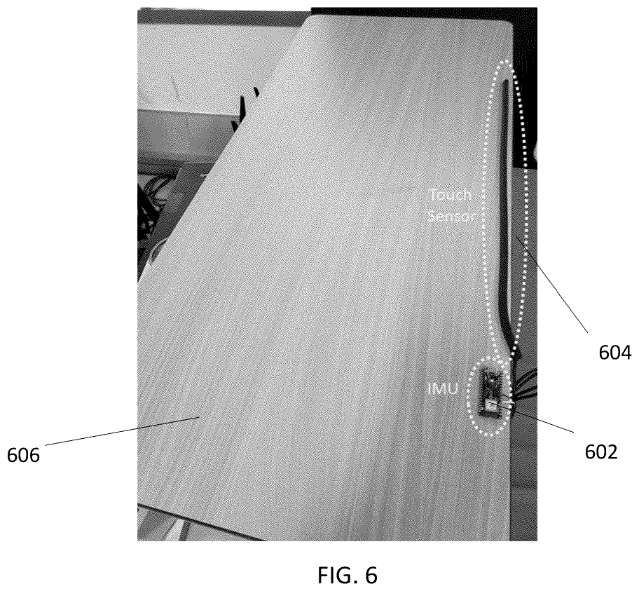

After the supporting device (for example chair/bed etc.) is already found in the previous step, by performing the “supporting surface detection” process, the supporting surface may then be detected by either of the following: locations or corners annotated in the system by the user or the operator of the system; AI training on the depth and/or point cloud image and AI recognition the bed surface automatically; and/or using the existing feature of the bed (for example the bed rail) and/or adding landmarks to the bed. Since their locations are known and can be found by the sensors and AI, the bed surface can then be determined. Referring to FIG. 6, sensors, such as IMU 602 and touch sensor 604 may be installed to the rails, and IMU may also be installed to the bed 606, such that the system may immediately generate a warning if these sensors are triggered.

In addition, if the supporting surface is a bed surface, a height sensor may be installed to measure the height of the bed surface. By analysing the point cloud data, the largest and/or the best fit horizontal plane at the measured height representing the bed surface. This can be double confirmed by comparing the area of the detected plane and area of the bed (pre-defined according to the bed model).

Then, by performing the “patient's posture and event detection” process, numerous depths and/or point cloud images of different posture of different people under different condition are used to perform AI training and the trained AI models can be used to recognise the postures. Alternatively, or additionally, the processing module may identify a position and/or a posture of the object based on a skeleton of the object.

Preferably, the processing module is arranged to identify a portion of the object being outside of the support surface to determine if the activity is risky based on a ratio of points in the point cloud representing the object staying on/above the support surface of the item of supporting furniture and outside of the support surface.

For example, the patient's posture can be found by using the trained AI models. Alternatively, or additionally, the patient's body part and/or skeleton can be found by using AI and the location of joints to find the patient posture. In addition, the processing module may further identify location of the object based on a head and/or a shoulder of the object, so as to evaluate the risk level of the activity.

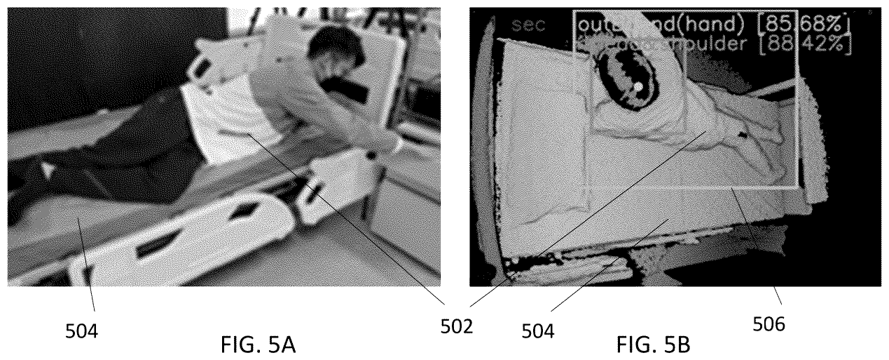

With reference to FIGS. 5A and 5B, the patient 502 is moving his hand out of the bed area 504 and recognised by the system (indicated by the box, the system is trained with similar pictures with different condition and persons). The head and shoulder are also recognised to identify the location of the patient. The percentage inside the bracket 506 is the confidence level of the detection. In addition, the duration of the posture detected is also recorded and displayed. In this example, such a posture, the activity of the patient reaching out extensively, may be identified as a risky activity.

Optionally or additionally, the processing module is arranged to predict the risky activity performed by the object with reference to a tracked posture of the object captured in a sequence of depth images provided by the 3D spatial sensors. Preferably, by tracking the posture of continuous frames and/or the sequence of the posture, the corresponding activity/event/intention can be modelled, predicted and detected.

Lastly, by performing the “status analysis and warning generation” process, firstly, the posture of the target object is extracted from the background. The patient's posture is recognised by the system using the aforesaid method, dangerous posture can be defined in the system and immediate warning can be generated, or if the duration of detecting posture is longer than a pre-set threshold (thresholds to different patient or target may be different depends on their profile or dynamically adjust their previous system history).

Preferably, in some example embodiments, the warning module is arranged to generate the alert upon detecting an activity performed by the object for a predetermined period of time.

In addition, the patient's activity/event is recognised by the system using the aforesaid method, dangerous activity/event can be defined in the system and immediate warning can be generated, or if the duration of detecting posture is longer than a pre-set threshold. According to different predicted events or activities, warning can be generated if certain status of the furniture is detected, for example if the rail of the medical bed is lowered down.

For example, after the bed surface, the patient's posture, and the point cloud representing the patient are found by the aforesaid methods. When the patient is moving outside the bed, the portion of his body and what body parts are outside the bed can by calculated. The followings are example events which may trigger a warning or an alert being generated.

Firstly, if the ratio of the numbers of point of the point cloud representing the patient outside the bed and the numbers of point of the point cloud representing the patient inside the bed is larger than a pre-set threshold, wherein the thresholds to different patients can be different depends on their profile or dynamically adjust their previous system history.

Secondly, combining the status of the furniture and the location of the furniture and the patient body part, the intent of the user can be defined. For example when the user if touching the closet, his hand is outside the bed, but the danger level is different if the user is just grabbing things on top of the closet or he is opening the closet.

Alternatively, warning can be generated if certain intent is detected, for example if patient is opening the closet, or warning can also be generated considering together with the patient's intent and the target, for example when the patient is moving his body outside the bed and the closet door is open.

Although not required, the embodiments described with reference to the figures can be implemented as an application programming interface (API) or as a series of libraries for use by a developer or can be included within another software application, such as a terminal or personal computer operating system or a portable computing device operating system. Generally, as program modules include routines, programs, objects, components and data files assisting in the performance of particular functions, the skilled person will understand that the functionality of the software application may be distributed across a number of routines, objects or components to achieve the same functionality desired herein.

It will also be appreciated that where the methods and systems of the present invention are either wholly implemented by computing system or partly implemented by computing systems then any appropriate computing system architecture may be utilized. This will include tablet computers, wearable devices, smart phones, Internet of Things (IoT) devices, edge computing devices, standalone computers, network computers, cloud-based computing devices and dedicated hardware devices. Where the terms “computing system” and “computing device” are used, these terms are intended to cover any appropriate arrangement of computer hardware capable of implementing the function described.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Any reference to prior art contained herein is not to be taken as an admission that the information is common general knowledge, unless otherwise indicated.

1. A method for monitoring activities of an object, comprising the steps of:
providing a depth image capturing at least a part of the object and an item of supporting furniture, wherein the item of supporting furniture is provided with a support surface arranged to physically support the object to be disposed thereon, and the object is movable relative to the item of supporting furniture;
processing the depth image to determine an activity of the object and the item of supporting furniture including the support surface being captured in the depth image; and
generating an alert upon a determination of the activity of the object being identified as a risky activity.

2. The method of claim 1, wherein the depth image is captured by a 3D spatial sensor includes stereo camera, 3D solid-state LiDAR, or structure light camera.

3. The method of claim 2, wherein the step of processing the depth image comprising the step of converting the depth image to point cloud data for further 3D analysis of the object and the item of supporting furniture so as to determine the activity of the object.

4. The method of claim 3, wherein the step of processing the depth image further comprising the step of identifying a location of the item of supporting furniture, including locating the support surface of the item of supporting furniture captured in the depth image.

5. The method of claim 4 wherein the step of identifying the location of the item of supporting furniture includes at least one of:
identifying one or more machine-detectable markers each indicating a predetermined position of a feature of the item of supporting furniture;
annotating the location of the item of supporting furniture by an operator; or
determining the location of the item of supporting furniture using AI image recognition.

6. The method of claim 4, wherein the step of processing the depth image further comprising the step of identifying a status of the item of supporting furniture and other furniture detectable by one or more sensor and/or computer vision.

7. The method of claim 4, wherein the step of processing the depth image further comprising the step of identifying a position and/or a posture of the object based on a trained AI models and/or skeleton of the object.

8. The method of claim 7, wherein the step of processing the depth image further comprising the step of predicting the risky activity performed by the object with reference to (i) a tracked posture of the object captured in a single and/or a sequence of depth images and/or (ii.) the status of the furniture other than the supporting furniture.

9. The method of claim 8, wherein the step of processing the depth image further comprising the step of identifying a portion of the object being outside of the support surface to determine if the activity is risky based on a ratio of points in the point cloud representing the object staying on/above the support surface of the item of supporting furniture and outside of the support surface.

10. The method of claim 1, wherein the object is a patient or an object requiring caregivers' and/or other people attentions.

11. A system of monitoring activities of an object, comprising:
an 3D spatial sensor arranged to provide a depth image capturing at least a part of the object and an item of supporting furniture, wherein the item of supporting furniture is provided with a support surface arranged to physically support the object to be disposed thereon, and the object is movable relative to the item of supporting furniture;
a processing module arranged to process the depth image to determine an activity of the object and the item of supporting furniture including the support surface being captured in the depth image; and
a warning module arranged to generate an alert upon a determination of the activity of the object being identified as a risky activity.

12. The system of claim 11, wherein the depth image is captured by a 3D spatial sensor includes stereo camera, 3D solid-state LiDAR, or structure light camera.

13. The system of claim 12, wherein the processing module is arranged to convert the depth image to point cloud data for further 3D analysis of the object and the item of supporting furniture so as to determine the activity of the object.

14. The system of claim 13, wherein the processing module is arranged to identify a location of the item of supporting furniture, including to locate the support surface of the item of supporting furniture captured in the depth image.

15. The system of claim 14, wherein the processing module is arranged to identify a location of the item of supporting furniture by performing at least one of:
identifying one or more machine-detectable markers each indicating a predetermined position of a feature of the item of supporting furniture;
annotating the location of the item of supporting furniture by an operator; or
determining the location of the item of supporting furniture using AI image recognition.

16. The system of claim 14, wherein the processing module is arranged to identify a status of the item of supporting furniture and other furniture detectable by one or more sensor and/or computer vision.

17. The system of claim 14, wherein the processing module is arranged to identify a position and/or a posture of the object based on a trained AI models and/or skeleton of the object.

18. The system of claim 17, wherein the processing module is arranged to predict the risky activity performed by the object with reference to (i) a tracked posture of the object captured in a single and/or a sequence of depth image(s) and/or (ii.) the status of the furniture other than the supporting furniture.

19. The system of claim 18, wherein the processing module is arranged to identify a portion of the object being outside of the support surface to determine if the activity is risky based on a ratio of points in the point cloud representing the object staying on/above the support surface of the item of supporting furniture and outside of the support surface.

20. The method of claim 11, wherein the object is a patient or an object requiring caregivers' and/or other people attentions.
